(19) United States
(12) Patent Application Publication
(10) Pub. No.: US 2018/0286430 A1
(43) Pub. Date: Oct. 4, 2018

(54) SPEECH EFFICIENCY SCORE

(71) Applicant: NINISPEECH LTD., Haifa (IL)

(72) Inventors: Yair SHAPIRA, Haifa (IL); Yoav MEDAN, Haifa (IL); Ofer AMIR, Kiryat Ono (IL)

(21) Appl. No.: 15/764,545

(22) PCT Filed: Oct. 5, 2016
(86) PCT No.: PCT/IL2016/051081

§ 371 Date: Mar. 29, 2018

Related U.S. Application Data
(60) Provisional application No. 62/239,303, filed on Oct. 9, 2015.

(51) Int. Cl. G10L 25/66; G10L 25/78; G10L 15/22; A61B 5/00; A61B 5/16

(52) U.S. Cl. CPC G10L 25/66; G10L 25/78; G10L 15/22; A61B 5/4803; A61B 2562/0204; A61B 5/4082; A61B 5/4088; A61B 5/7282; A61B 5/165

(57) ABSTRACT

The present disclosure provides methods, devices and systems for assessing/evaluating the verbal fluency of a user by obtaining a speech (audial/acoustic signal) from a user, detecting disrupted/stuttered and fluent speech time-intervals in the speech, calculating a Disrupted-time value and Fluent-time value based on the disrupted/stuttered and fluent speech time-intervals respectively, and deriving a speech efficiency score for the user/speech based on the Disrupted-time value and Fluent-time value.

### TECHNICAL FIELD

The present disclosure generally relates to the field of speech fluency evaluation.

### BACKGROUND

Speech fluency conditions such as stuttering and cluttering may impose difficulties on the lifestyles and self-esteem of people suffering from them. While there are various methods of treating such conditions, the metrics for assessing the severity of the conditions and evaluating the fluency of speech remain insufficiently developed.

Some existing metrics for speech fluency evaluation include methods such as the “Lewis-Sherman” scale, a “percentage of syllables stuttered”, stuttering events per minute, “Iowa scale” and Stuttering Severity Instrument (SSI). Common to these methods is that they are subjective, highly variable between judges, controversial, measured manually (therefor require time consuming labor) and are based on clinic-recording instead of speech in the, real world, daily routine of the speaker.

There is thus a need in the art for speech measurement that will provide consistent, useful and objective indication of speech fluency.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiments, there are provided herein devices, systems and methods for providing a speech efficiency evaluation/assessment, for example by providing a speech efficiency score (SES). It is well known that speech is used for transferring information. If a speaker cannot transfer new information, the listener is typically annoyed or tends to lose patience. According to some embodiments, a speech efficiency evaluation, as disclosed herein measures a ratio of time in which the speaker is actually transmitting information, for example, new information. In accordance with some embodiments, contrary to currently used speech measurements, the SESs disclosed herein focus on the essence of fluency or lack of fluency (disfluency).

According to some embodiments, the SES is objective, automatically calculated/obtained and consistent. According to some embodiments, SES measurements, as disclosed herein, can operate on real-world data, in other words, on a speaker's every-day speaking and not necessarily at the clinician's office.

According to some embodiments, there are provided herein devices, systems and methods for speech fluency assessment/evaluation by detecting and measuring disfluent speech time-interval(s) in a speech, detecting fluent speech time-interval(s) in the speech, and deriving a speech efficiency score based on the disfluent speech time interval(s) and the fluent speech time-interval(s).

Advantageously, a speech efficiency score based on stuttered and fluent time intervals may provide an objective assessment of speech fluency and speech conditions, and facilitate quantifiable measurements for availing a reliable tracking of the condition/fluency.

According to some embodiments, the speech efficiency score may be utilized for evaluating and assessing the effectiveness of a speech treatment or exercise. Advantageously, evaluating the effectiveness of a treatment or exercise may enable varying the treatment or exercise to achieve an improved fluency per user or a plurality of users.

According to some embodiments, the speech efficiency score may be utilized for diagnosing speech-related disabilities/conditions. According to some embodiments, the speech efficiency score may be utilized for detecting neurological disorders/conditions, for example neurodegenerative conditions (such as Amyotrophic lateral sclerosis, Parkinson's, Alzheimer's, Huntington and others).

According to some embodiments, the speech efficiency score may be utilized for enhancing the speech efficiency of general speakers, and not necessarily due to a known condition or a detection or diagnostic of a condition.

According to some embodiments, the speech efficiency score may be utilized for enhancing the speech efficiency of professionals, such as public speakers, entertainers, diplomats, sales and marketing professionals and the like.

According to some embodiments, there is provided a device for speech fluency assessment/evaluation, including an acoustic sensor, configured to convert sound into an electrical signal, and a processing circuitry, configured to determine a speech period; obtain, from the acoustic sensor, an electrical signal of speech within the speech period, detect a disfluent speech time-interval(s) in the speech period, and calculate a disfluent-time value based thereon, detect a fluent speech time-interval(s) in the speech period, and calculate a fluent-time value based thereon, and derive a speech efficiency score of the speech period based on the fluent-time value and the disfluent-time value.

According to some embodiments, the processing circuitry is further configured to detect a quiet time-interval(s) in the speech period, subtract/remove the detected quiet time interval(s) from the speech period to obtain an active speech time-interval(s) in the speech period, and calculate the fluent-time value, calculate the disfluent-time value and derive the speech efficiency score within the active speech time-interval(s) of the speech period.

According to some embodiments, the processing circuitry is further configured to categorize the speech efficiency score based on predetermined categorization criteria.

According to some embodiments, deriving a speech efficiency score includes dividing the fluent-time value by the sum of the fluent-time value and disfluent-time value and assigning the result to a speech efficiency score (SES) metric.

According to some embodiments, deriving a speech efficiency score includes dividing the disfluent-time value by the sum of the fluent-time value and disfluent-time value and assigning the result to a speech inefficiency score (SIES) metric.

According to some embodiments, deriving a speech efficiency score includes dividing the fluent-time value by the disfluent-time value and assigning the result to a fluent to disfluent ratio (FTDR).

According to some embodiments, detecting a disfluent speech time-interval(s) in the speech period includes detecting a time-interval in the speech period in which there is an unnecessary/redundant repetitiveness of a sound, syllable, part of a word, word and/or phrase.

According to some embodiments, detecting a disfluent speech time-interval(s) in the speech period includes detecting a time-interval that includes an intermittent vocal utterance or interjection.

According to some embodiments, detecting a disfluent speech time-interval(s) in the speech period includes detecting a time-interval that includes an abrupt vocal utterance.

According to some embodiments, detecting a disfluent speech time-interval(s) in the speech period includes detecting a time-interval that includes a prolongation having a duration that exceeds a predetermined threshold.

According to some embodiments, detecting a disfluent speech time-interval(s) in the speech period includes detecting a time-interval that includes blocking of speech.

According to some embodiments, the processing circuitry is further configured to convert the electrical signal of speech to a frequency domain and to detect a disrupted/stuttered speech time-interval(s) in the speech period by analyzing the electrical signal in the frequency domain.

According to some embodiments, the processing circuitry is further configured to calculate a progression score by comparing the derived speech efficiency score with a reference speech efficiency.

According to some embodiments, the processing circuitry is configured to perform an offline analysis, such that the steps of detecting the disfluent speech time-interval(s), calculating the disfluent-time value, detecting the fluent speech time-interval(s), calculating the fluent-time value, and deriving a speech efficiency score of the speech period, are performed after the speech period is expired.

According to some embodiments, the processing circuitry is configured to perform an online analysis, such that the steps of detecting the disfluent speech time-interval(s), calculating the disfluent-time value, detecting the fluent speech time-interval(s), calculating the fluent-time value, and deriving a speech efficiency score of the speech period, are at least partially performed before the speech period is expired.

According to some embodiments, the device further includes a user interface unit configured to provide the user with information related to a speech.

According to some embodiments, the user is a speaker and/or a practitioner.

According to some embodiments, the processing circuitry is configured to derive a speech efficiency score of the speech period by dividing the fluent-time value with the sum of the fluent-time value and disfluent-time.

According to some embodiments, there is provided a speech fluency assessment/evaluation method, including determining a speech period, obtaining an electrical signal of speech within the speech period, detecting a disfluent speech time-interval(s) in the speech period, and calculating a disfluent-time value based thereon, detecting a fluent speech time-interval(s) in the speech period, and calculating a fluent-time value based thereon, and deriving a speech efficiency score of the speech period based on the fluent-time value and the disfluent-time value.

According to some embodiments, the method further includes detecting an active speech time-interval(s) in the speech period, and calculating the Fluent-time value, calculating the disfluent-time value and deriving the speech efficiency score within the active speech time-interval(s) of the speech period.

According to some embodiments, the method further includes categorizing the speech efficiency score based on predetermined categorization criteria.

According to some embodiments, detecting a disfluent speech time-interval(s) in the speech period includes detecting a time-interval in the speech period in which there is a repetitiveness of a character.

According to some embodiments, the detecting a disfluent speech time-interval(s) in the speech period includes detecting a time-interval that includes an intermittent vocal utterance.

According to some embodiments, the detecting a disfluent speech time-interval(s) in the speech period includes detecting a time-interval that includes an abrupt vocal utterance.

According to some embodiments, the method further includes calculating a progression score by comparing the derived speech efficiency score with a reference speech efficiency.

According to some embodiments, detecting the disfluent speech time-interval(s), calculating the disfluent-time value, detecting the fluent speech time-interval(s), calculating the fluent-time value, and deriving a speech efficiency score of the speech period. are performed after the speech period is expired.

According to some embodiments, detecting the disfluent speech time-interval(s), calculating the disfluent-time value, detecting the fluent speech time-interval(s), calculating the fluent-time value, and deriving a speech efficiency score of the speech period, are at least partially performed before the speech period is expired.

According to some embodiments, the method further includes providing a user with information related to a speech.

According to some embodiments, the user is a speaker and/or a practitioner.

According to some embodiments, deriving a speech efficiency score of the speech period includes dividing the fluent-time value with the sum of the fluent-time value and disfluent-time.

According to some embodiments, the speech efficiency score includes a speech inefficiency score (SIES) and the method further includes deriving a speech inefficiency score by dividing the disfluent-time value with the sum of the fluent-time value and disfluent-time.

According to some embodiments, the speech efficiency score includes a fluent to disfluent ratio (FTDR) and the method further includes deriving a fluent to disfluent ratio by dividing the fluent-time value with the disfluent-time value.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1a and FIG. 1b schematically illustrate a detection of stuttered and speech time intervals, according to some embodiments;

FIG. 2 schematically illustrates a method for deriving a speech efficiency score, according to some embodiments;

FIG. 3 schematically illustrates a method for deriving a speech efficiency score, according to some embodiments;

FIG. 4 schematically illustrates a system for deriving a speech efficiency score, according to some embodiments;

FIG. 5 schematically illustrates a learning system for deriving a speech efficiency score, according to some embodiments,

FIG. 6 schematically illustrates a speech pattern including prolongation, according to some embodiments;

FIG. 7 schematically illustrates a speech pattern including repetition, according to some embodiments;

FIG. 8 schematically illustrates a speech pattern including interjection, according to some embodiments; and

FIG. 9 schematically illustrates a speech pattern including block time intervals, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present invention may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of non-transitory memory media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

According to some embodiment, there are provided herein devices, systems and methods for speech fluency assessment/evaluation by detecting and measuring disfluent speech time-interval(s) in a speech, detecting fluent speech time-interval(s) in the speech, and deriving a speech efficiency score based on the disfluent speech time interval(s) and the fluent speech time-interval(s).

Advantageously, a speech efficiency score based on time intervals of disfluent and fluent time intervals may provide an objective assessment of speech fluency and speech conditions, and facilitate quantifiable measurements for promoting a reliable tracking of the condition/fluency.

According to some embodiments, the speech efficiency score may be utilized for evaluating and assessing the effectiveness of a speech treatment or exercise. Advantageously, evaluating the effectiveness of a treatment or exercise may enable varying the treatment or exercise for achieving an improved fluency per user or a plurality of users.

According to some embodiments, the speech efficiency score may be utilized for diagnosing speech-related disabilities/conditions. According to some embodiments, the speech efficiency score May be utilized for diagnosing/detecting neurological disorders/conditions, for example neurodegenerative conditions (such as Amyotrophic lateral sclerosis, Parkinson's, Alzheimer's, Huntington and others). According to some embodiments, the speech efficiency score may be utilized for diagnosing/detecting psychological conditions, such as depression, anxiety and others. According to some embodiments, the speech efficiency score may be utilized for diagnosing/detecting mental conditions or disorders such as dyslexia, autism, hyperactivity and others.

From a listener/receiver standpoint, a speech lasts for a certain period of time-speech period. During this period of time, there may be time intervals in which the speech is fluent, other time intervals in which the speech is disfluent, and quiet/silence time intervals. According to some embodiments, the speech efficiency is evaluated by the ratio of the fluent speech time intervals from the total time period of the speech. Accordingly, the speech efficiency score may be measured based on the accumulative duration of the fluent speech time intervals, and the ratio thereof from the total speech time.

According to some embodiments, the derived speech efficiency score is based on the total time of fluent speech and the total time of disfluent speech in a speech period of the user. According to some embodiments, the severity of the stuttering condition is measured by the total amount of time of disfluency in comparison to, or as a portion of the net speech time. According to some embodiments, the net speech time may be derived by subtracting the quiet/silence time periods/intervals from the total time of the speech. According to some embodiments, the severity of the stuttering condition is measured by the total amount of time of disfluency in comparison to, or as a portion of the total amount of fluent speech time.

According to some embodiments, the disfluent time intervals are considered noise intervals, and little/no information may be obtained from these intervals, while fluent speech time intervals are considered data intervals, and information may be obtained from these intervals. According to some optional embodiments, the ratio between the duration of the noise intervals and the duration of the data intervals may determine the severity of the stuttering/speech-condition.

According to some embodiments, the speech period may include silent/empty time intervals, and during these intervals little/no speech is detected. According to some embodiments, the silent/empty intervals may be at least partially removed/subtracted from the total speech time. According to some embodiments, the silent/empty intervals may be at least partially considered stuttering intervals. According to some embodiments, the silent/empty intervals may be at least partially considered fluent speech intervals.

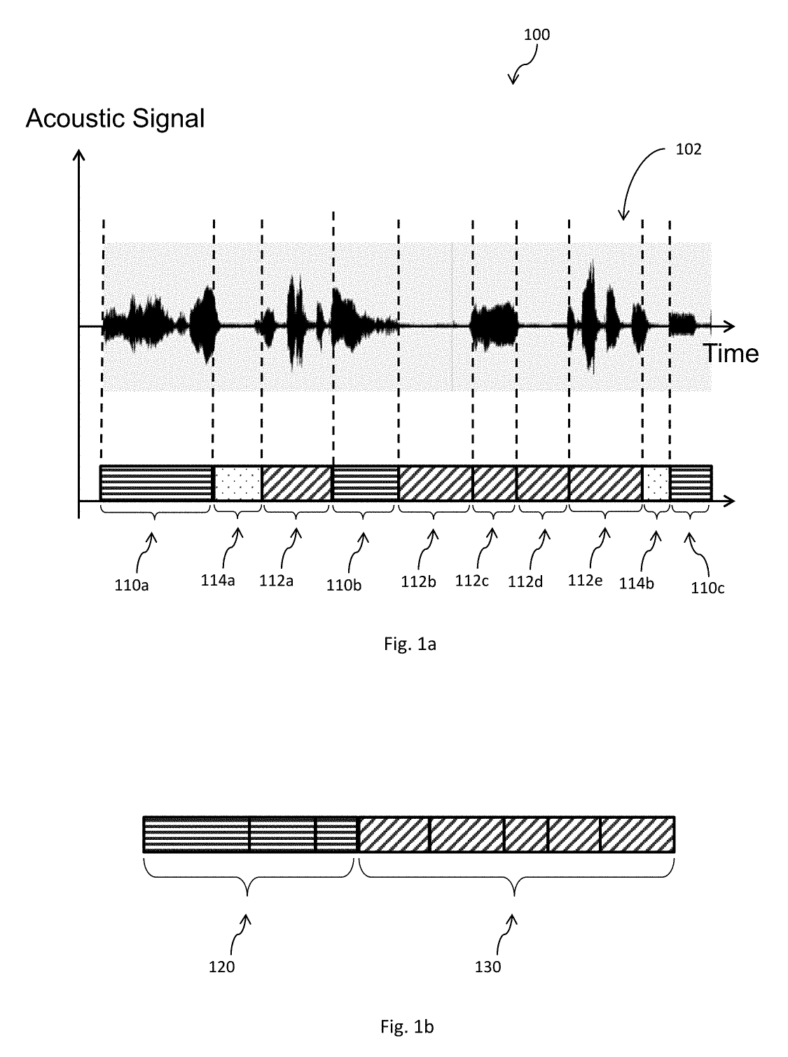

Reference is now made to FIG. 1a and FIG. 1b, which schematically illustrate detection 100 of disfluent and fluent time intervals in a speech period 102, according to some embodiments. According to some embodiments, speech period 102 may be received from a user, or determined by the device/system. According to some embodiments, speech period 102 is analyzed to detect fluent speech time intervals, such as fluent intervals 110a, 110b, and 110c, and disfluent speech intervals, such as disfluent intervals 112a, 112b, 112c, 112d and 112e. Additionally, the analysis may also detect silent time intervals, such as silent intervals 114a and 114b.

As illustrated, various disfluent intervals may be identified by detecting different characteristics. For example, disfluent intervals 112a and 112e are identified by detecting abrupt intermittency of an utterance, disfluent intervals 112b and 12d are identified by detecting prolonged “block” quiet/silent periods, and disfluent interval 112c is identified by detecting a prolonged utterance.

According to some embodiments, the total time duration of fluent intervals 110a, 110b, and 110c may be calculated by summing up the durations thereof, and a fluent-time value 120 may be assigned based on the total calculated duration. Additionally, according to some embodiments, the total time duration of disfluent intervals 112a, 112b, 112c, 112d and 112e may be calculated by summing up the durations thereof, and a disfluent-time value 130 may be assigned based on the total calculated duration.

According to some embodiments, if fluent-time value 120 is A, and disfluent-time value 130 is B, then the speech efficiency score (SES) may be calculated by dividing A by A+B:

SES=A/(A+B)

According to some embodiments, if fluent-time value 120 is A, and disfluent-time value 130 is B, then a speech inefficiency score (SIES) may be calculated by dividing B by A+B:

SIES=B/(A+B)

According to some embodiments, if fluent-time value 120 is A, and disfluent-time value 130 is B, then a fluent-to-disfluent ratio (FDFR) may be calculated by dividing A by B:

FDFR=A/B

As used herein, and according to some embodiments, the term “speech efficiency score” or “SES” may be interchangeable with one or more of the scores: SIED and/or FDFR.

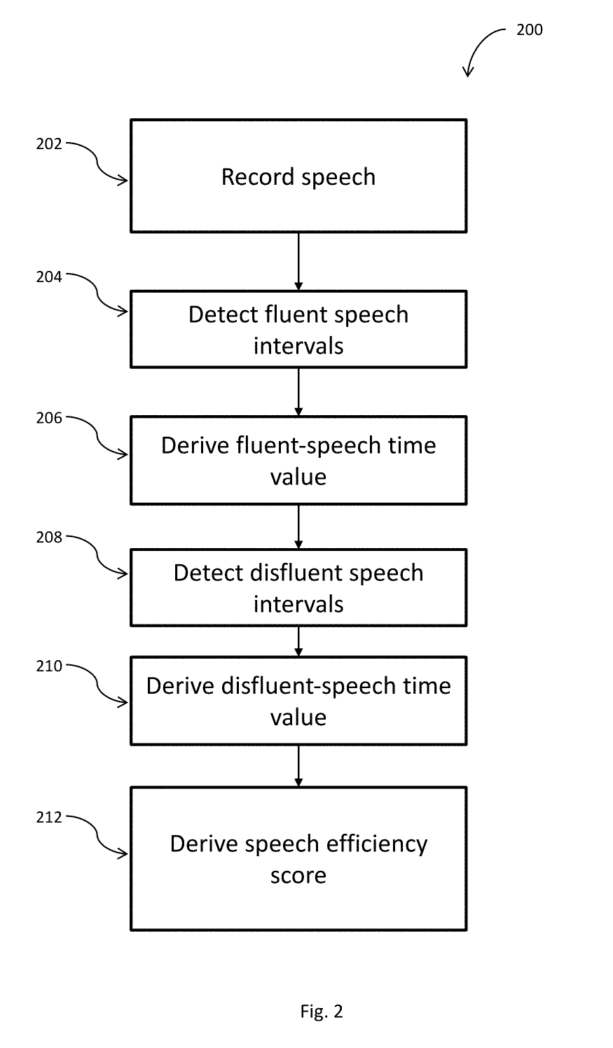

Reference is now made to FIG. 2, which schematically illustrates a method 200 for deriving a speech efficiency score, according to some embodiments. According to some embodiments, method 200 begins by recording a speech (step 202) using an acoustic sensor such as a microphone. Then (or in other embodiments, simultaneously while the speech is being captures/obtained), fluent speech time intervals are detected (step 204), and a fluent-speech time value is derived (step 206). Additionally, disfluent speech time intervals are detected (step 208), and a disfluent speech time value is derived (step 210). Finally, a speech efficiency score may be derived (step 212) based on the derived disfluent speech time value and fluent-speech time value.

According to some embodiments, silence/quiet time intervals are also detected and a silence/quiet time value is derived.

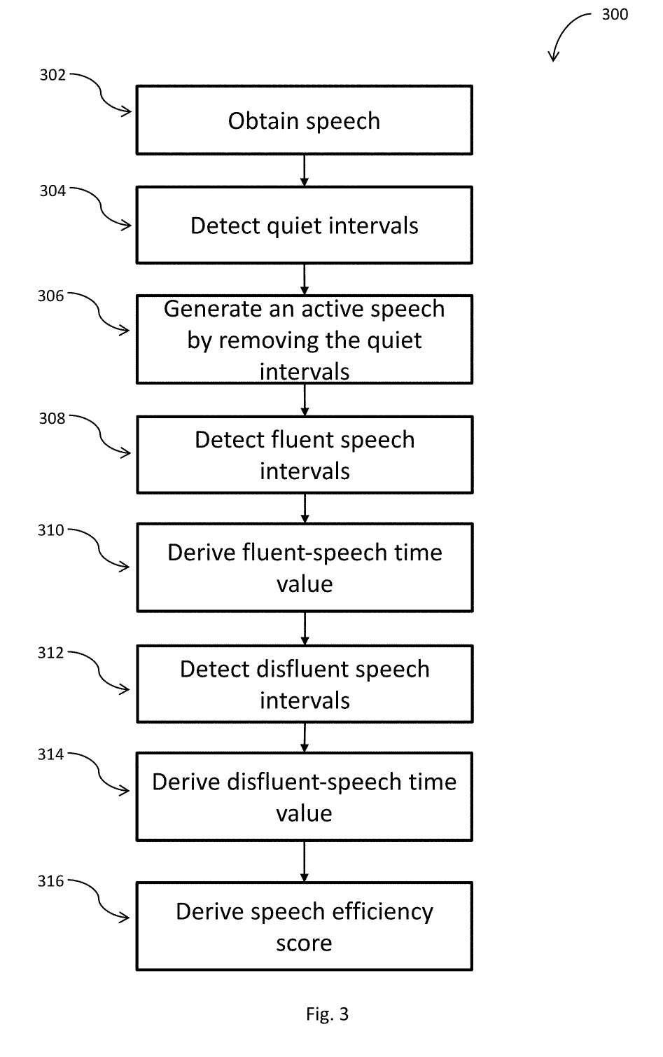

Reference is now made to FIG. 3, which schematically illustrates a method 300 for deriving a speech efficiency score including quiet period(s) detection, according to some embodiments. According to some embodiments, method 300 begins by obtaining a speech signal (step 302), which may be an offline speech signal or an online speech signal, then quiet intervals are detected (step 304), for example by detecting periods of silence within the speech signal that exceed a threshold, then an active speech signal may be generated by eliminating/removing the quiet intervals (step 306). The active speech is further analyzed for detection of fluent speech intervals (step 308) and a fluent speech time value is derived based thereon (step 310), and detection of disfluent speech intervals (step 312) and deriving a disfluent time value based thereon (step 314). Afterwards, a speech efficiency score may be derived (step 316) based on the fluent speech time value and the disfluent speech time value.

According to some embodiments, the speech is recorded and provided for offline analysis and derivation of a speech efficiency score. According to some embodiments, the speech is at least partially directly streamed for online analysis.

As used herein, the term offline analysis may refer to an analysis on a speech that was recorded prior to the analysis. An example of an offline analysis may be an analysis done by a computing/processing unit on a speech recording provided by a speaker, by a caregiver, or by a professional clinician as an electronic file, such as an audio file. According to some embodiments, the audio file may be encrypted, compressed and/or formatted. According to some embodiments, the format type may be uncompressed, lossless-compressed or Lossy compressed. According to some embodiments, the audio file format may be an mp3, aiff, aac, 3gp, amr, dct, su, dss, dvf, flac, gsm, m4p, m4a, mmf, mpc, msv, ogg, oga, opus, raw, tta, sln, vox, way, wma, wv, webm or the like. According to some embodiments, the device/system may include a decompressor/decoder configured to decompress/decode the audio file.

As used herein, the term online analysis may refer to an analysis on a speech as it is being provided or vocalized by the user. According to some embodiments, the online analysis is a real-time analysis. According to some embodiments, the online analysis is a non-real-time analysis.

According to some embodiments, the analysis is done locally, for example by a local computer and/or mobile device. According to some embodiments, the analysis is done remotely, for example by a server. According to some embodiments, the server may include a cloud server.

According to some embodiments, the analysis may be automatic, and initiated without the immediate actuation of the user, for example, a mobile device such as a smart wearable device or a smart phone may detect a speech of the user and analyze or record it automatically. According to some embodiments, the device/system may detect that a certain audial feature is associated with a certain user by utilizing a speech recognition algorithm. According to some embodiments, the device may obtain speech signals/periods by recognizing the speech periods of the user during phone calls.

According to some embodiments, a speech efficiency score may be provided to the user after the end of the speech part. According to some embodiments, a dynamic speech efficiency score may be provided to the user even during the speech part.

According to some embodiments, the systems/devices may further facilitate speech training sessions for improving the speech efficiency score of the user. According to some embodiments, the speech training sessions are generated or provided based on the derived speech efficiency score of the user.

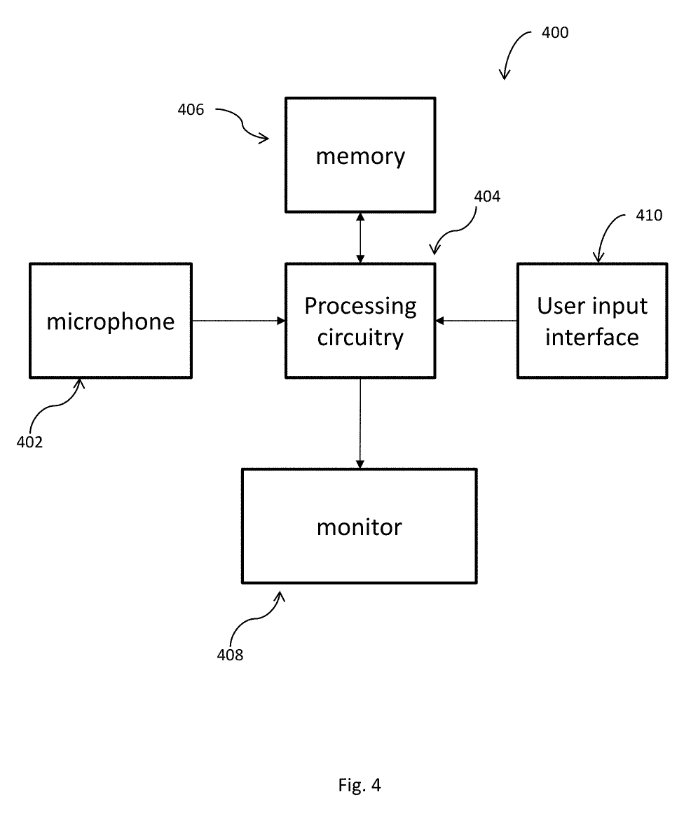

Reference is now made to FIG. 4, which schematically illustrates a system 400 for deriving a speech efficiency score, according to some embodiments. According to some embodiments, system 400 may include an acoustic sensor, such as microphone 402, which is configured to sense acoustic signals and convert them to an electric signal to be provided to a controller and analyzer, such as processing circuitry 404, which is configured to analyze the electric signal(s) obtained from microphone 402 for detecting and measuring intervals of disfluent and fluent speech within a speech period. Processing circuitry 404 may then provide the user with a derived speech efficiency score via a user feedback/training interface such as monitor 408. According to some embodiments, processing circuitry 404 may be communicatively connected to a memory device 406 which may include instruction memory segments configured for storing command code for operating the system to derive the speech efficiency score. According to some embodiments, memory device 406 may further include data segments for storing additional information such as user information, disfluency patterns information, history information, speech training sessions, user progress, speech efficiency scores or the like.

According to some embodiments, processing circuitry 404 may further be connected to a user input interface 410 for obtaining control and information from the user. The control may include initiation and termination signals, session duration signal or the like. The information may include user gender, age, profession, hobby and the like. According to some embodiments, user input reference 410 may include a touch interface, a keyboard, a computer mouse, a camera or the like.

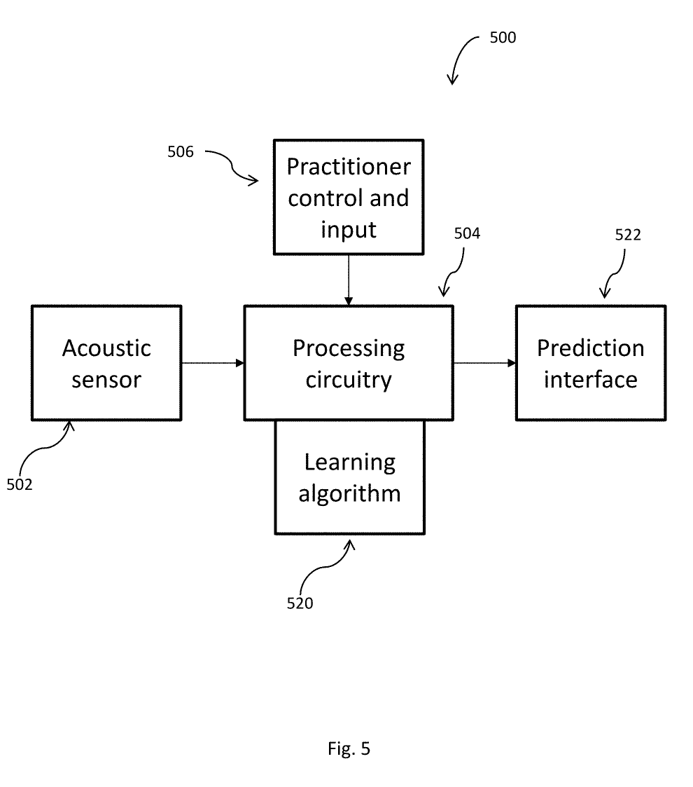

Reference is now made to FIG. 5, which schematically illustrates a learning system 500 for deriving a speech efficiency score, according to some embodiments. System 500 may include an acoustic sensor 502 configured to sense audial/acoustic speech and transform it to an electric signal to be delivered to a processing circuitry 504. According to some embodiments, processing circuitry 504 is configured to utilize a learning algorithm 520 for producing predictions of stuttering interval detection in the electric signal provided by acoustic sensor 502. The predictions may then be delivered to a prediction interface 522 and a practitioner would then examine the prediction and provide learning feedback to processing circuitry 504 via a control and input unit 506 for correcting the prediction or upholding it. According to some embodiments, learning algorithm 520 may include a neural structure machine learning architecture. According to some embodiments, learning algorithm 520 may include deep-learning machine architecture. According to some embodiments, learning algorithm 520 may include a genetic algorithm, similarity and metric learning, reinforcement learning, Bayesian networks, clustering, representation learning, association rule learning, decision tree learning, inductive logic programming, support vector machine, clustering or the like or any combination thereof:

According to some embodiments, there is provided a data structure including a first segment of information configured for storing a duration value of a disfluent time interval, and a second segment of information assigned for storing a duration of a fluent time interval. According to some embodiments, the data structure further includes a third segment of information assigned for storing a duration of quiet time interval. According to some embodiments, there is provided a data structure having an information segment configured for storing a speech efficiency score based on the durations of at least one fluent time interval and, if exists, at least one disfluent interval.

As used herein, the term stuttering, disfluency or speech conditions may refer to speech with involuntary repetition of sounds. According to some embodiments, the repetition of sounds is a repetition of a consonant, vowel, syllable, part of a word, word, or phrase. Stuttering may be referred to as a speech disorder in which the flow of speech is disrupted by involuntary prolongations of sounds, syllables, words or phrases as well as involuntary silent pauses or blocks in which the person who stutters is unable to produce sounds. Stuttering may also include abnormal hesitation or pausing before speech that may be referred to as blocks.

According to some embodiments, stuttering may be identified by detecting repeated movements such as syllable repetition, incomplete syllable repetition or multi-syllable repetition. According to some embodiments, stuttering may be measured by detecting fixed postures, with audible airflow (such as prolongation of a sound) or without audible airflow (such as a block of speech or a tense pause wherein no speech occurs, despite effort). According to some embodiments, stuttering may be measured by detecting superfluous speech which may be verbal (such as an interjection as an unnecessary “uh” or “urn” or as revisions) or non-verbal.

As used herein, a disfluent time interval may be defined as intervals that may be omitted from the speech to obtain a fluent speech. According to some embodiments, a disfluent time interval may include time intervals of blocks. According to some embodiments, a disfluent time interval may include time intervals of unnecessary repetition of sounds. According to some embodiments, a disfluent time interval may include time intervals of overly prolonged syllables. According to some embodiments, a disfluent time interval may include time intervals of interjections. According to some embodiments, a disfluent time interval may include time intervals of the silence periods on one or both sides of a repetition or interjection.

As used herein, the term speech interval may refer to a time interval that includes information, the omission of which may impair the fluency or information of the speech. According to some embodiments, a speech interval may include normal silence periods or pauses that may occur between words and/or sentences.

As used herein, the terms quiet/tare/silence time(s) and/or interval(s) may refer to intervals vacant of speech. According to some embodiments, quiet intervals occur as a result of obtaining audial signals even when no speech is intended such as in continuous recording.

According to some embodiments, disfluency detection may be achieved by comparing speech segments to known disfluency patterns and evaluating the similarities therebetween. According to some embodiments, disfluency detection may be achieved by utilizing a speech recognition algorithm for converting the recorded/streamed speech into text, and the intervals of the speech that do not get recognized by the speech recognition algorithm may be referred to as stuttering intervals.

Quiet-Time Intervals:

According to some embodiments, a quiet interval may refer to a silent interval, which is not a part of the fluent or disfluent speech. For example, during a dialog, when the second person speaks, is a quiet interval for the first person. According to some embodiments, pauses between words and sentences, and silence periods associated with disfluency, are not quiet-time intervals.

According to some embodiments, detecting quiet-time intervals may be done as follows: if period Q is a continuous period without meaningful speech, which is longer than some threshold duration, it may be considered as a quiet time interval. According to some embodiments, the threshold duration can be dynamic, for example the 2nd positive standard deviation of continuous silence periods, or the threshold duration can be predetermined.

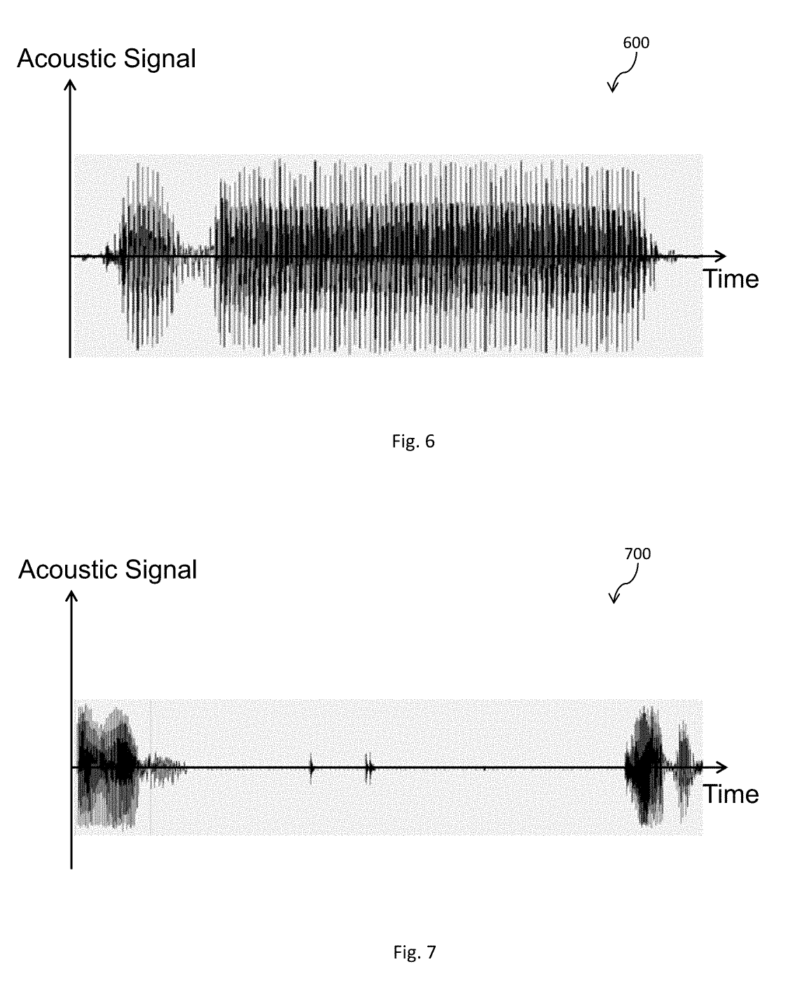

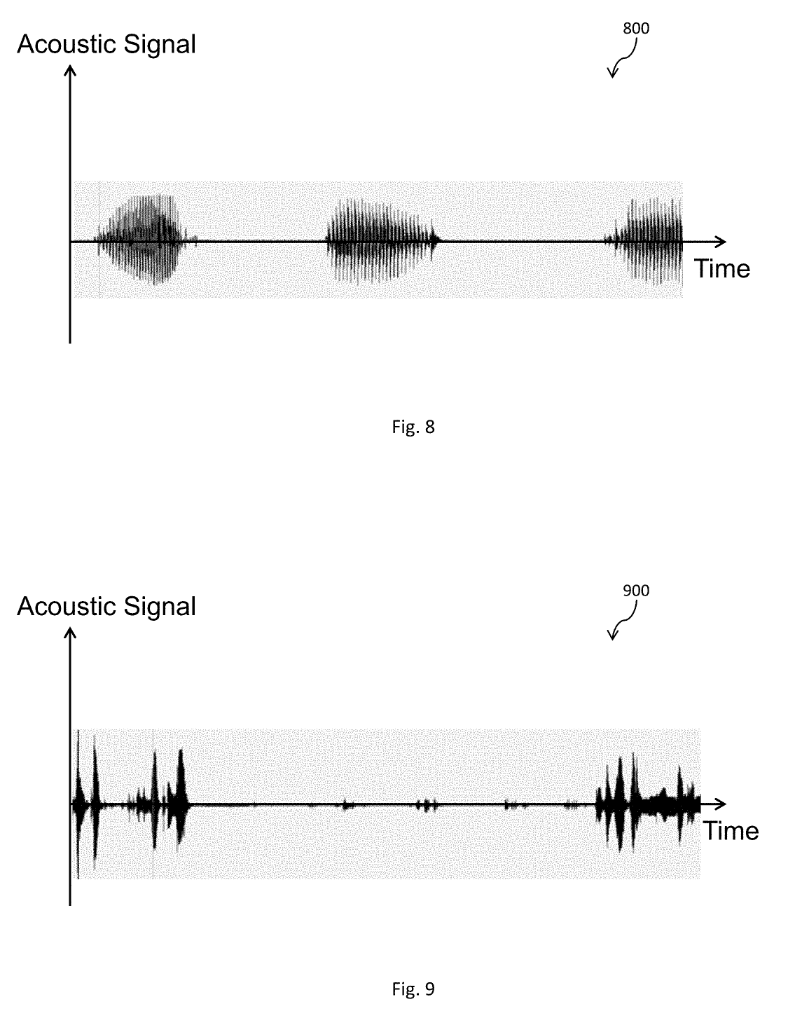

According to some embodiments, disfluent speech patterns and/or disfluent time intervals may include one or more of the following:
Prolongation: A prolonged sound is a continuous sound, which is significantly longer than the average duration of similar sounds. The average duration is dynamic, thus should be adapted to the language, speaker, and condition. The term “significantly longer” can mean, for example, longer than the 2^{nd} positive standard deviation of duration of similar sounds. FIG. 6 schematically illustrated prolongation 600, according to some embodiments.
Repetition: sounds that are involuntarily repeated, and bear no additional information. Such sounds may comprise of a consonant, vowel, and syllable, part of a word, word or phrase. Often repetitions are preceded and/or followed by silences, which may be considered part of the disfluent-time interval as well. FIG. 7 schematically illustrated prolongation 700, according to some embodiments.
Interjection: an interjection is a speech element that bears no information. It fills a gap, and is sometimes used by people with fluency conditions to fill blocks. The specific utterance may vary between speakers and languages (e.g. English-speakers often use “like” or “ok”, whereas Japanese use “ano”, and Chinese use “nega”). Often interjections are preceded and/or followed by silences, which may be considered part of the disfluent-time interval as well. FIG. 8 schematically illustrated interjection 800, according to some embodiments.
Block: blocks are silence periods, which are not part of the fluent speech. Blocks are often a result of the speaker trying but failing to produce sound. Other occurrences may be blocks in which the speaker takes excessively extra time to continue speech. FIG. 9 schematically illustrated block time intervals 900, according to some embodiments.
Disfluent time intervals: are intervals in which the above patterns are detected, including silence periods between them, which are not quiet-time intervals.

According to some embodiments, the detection of disfluent time intervals may be achieved by segmenting the active speech time period or the active speech to a plurality of segments, and comparing the patterns of each segment to a known pattern of disfluent speech. According to some embodiments, the segmentation may be a fixed-time segmentation. According to some embodiments, the segmentation may be based on pattern changes within the speech time period.

According to some embodiments, after obtaining a speech efficiency score, the result may then be categorized according to categorization criteria. According to some embodiments, the categorization criteria may include thresholds indicative of the severity of a speech conditions. According to some embodiments, the categorization criteria may include categories such as “excellent”, “good”, “fair”, “slightly disfluent”, “fluent”, “severely disfluent”, and the like, or any combination thereof.

As used herein, the term “speech period” may refer to a time period during which a speech is/was delivered. According to some embodiments, a speech period may be a phone-call conversation or a recording thereof. According to some embodiments, a speech period may be initiated and terminated (indicated) automatically. According to some embodiments, a speech period may be initiated and terminated (indicated) manually by a user, speaker, practitioner or others. According to some embodiments, a speech period may include quiet tine intervals. According to some embodiments, a speech period may include active speech periods or time interval(s).

As used herein, the term “active speech”, may refer to periods in which a speaker may be actively speaking or trying to speak or convey information. According to some embodiments, active speech may include fluent speech and/or disfluent speech. According to some embodiments, active speech may include “soundless periods” of speech that may be considered a part of fluent speech, such as soundless periods between sentences, or disfluent speech such as soundless stuttering blocks. According to some embodiments, soundless periods that are either pert of a fluent speech or a disfluent speech may be considered in the derivation of the speech efficiency score, while other quiet time intervals may be excluded in the derivation, such quiet time intervals may exist for example when the speech is a dialog and the current speaker is not the user.

As used herein, the term “active speech time-interval”, may refer to a time period, during which active speech occurs.

As used herein, the term “disfluent speech” may refer to speech in which no information is delivered despite the intention of delivering information through speaking. According to some embodiments, disfluent speech may include stuttering.

As used herein, the term or “disfluent speech time interval” may refer to a time period, during which disfluent speck occurs.

As used herein, the term “disfluent-time value”, may refer to a value indicative of a duration of a disfluent speech time interval or a plurality of disfluent time intervals. According to some embodiments, the disfluent-time value may include the total duration of disfluent speech time intervals. According to some embodiments, the disfluent-time value may include the ratio of the total duration of disfluent speech time intervals from the speech period and/or active speech period.

As used herein, the term “fluent speech” may refer to speech in which information is delivered fluently through speaking. According to some embodiments, fluent speech is vacant of disfluent speech and/or does not include stuttering.

As used herein the term “fluent speech time-interval” may refer to a time period, during which fluent speech occurs.

As used herein, the term “fluent-time value”, may refer to a value indicative of a duration of a fluent speech time interval or a plurality of fluent speech time intervals. According to some embodiments, the fluent-time value may include the total duration of fluent speech time intervals. According to some embodiments, the fluent-time value may include the ratio of the total duration of fluent speech time intervals from the speech period and/or active speech period.

As used herein, the term “speech efficiency score”, may refer to a metric for measuring the efficiency of speech. According to some embodiments, the speech efficiency score is indicative of the ratio between the fluent speech time and the total speech time (or active speech time).

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1.-32. (canceled)

33. A device for speech fluency assessment/evaluation, comprising:
an acoustic sensor, configured to convert sound into an electrical signal; and
a processing circuitry, configured to:
determine a speech period;
obtain, from said acoustic sensor, an electrical signal of speech within the speech period;
detect a disfluent speech time-interval(s) in the speech period, and calculate a disfluent-time value based thereon;
detect a fluent speech time-interval(s) in the speech period, and calculate a fluent-time value based thereon; and
derive a speech efficiency score of the speech period based on the fluent-time value and the disfluent-time value.

34. The device of claim 33, wherein said processing circuitry is further configured to:
detect a quiet time-interval(s) in the speech period;
subtract/remove the detected quiet time interval(s) from the speech period to obtain an active speech time-interval(s) in the speech period; and
calculate the fluent-time value, calculate the disfluent-time value and derive the speech efficiency score within the active speech time-interval(s) of the speech period.

35. The device of claim 33, wherein said processing circuitry is further configured to:
categorize the speech efficiency score based on predetermined categorization criteria.

36. The device of claim 33, wherein deriving a speech efficiency score comprises dividing the fluent-time value by the sum of the fluent-time value and disfluent-time value and assigning the result to a speech efficiency score (SES) metric.

37. The device of claim 33, wherein deriving a speech efficiency score comprises dividing the disfluent-time value by the sum of the fluent-time value and disfluent-time value and assigning the result to a speech inefficiency score (SIES) metric.

38. The device of claim 33, wherein deriving a speech efficiency score comprises dividing the fluent-time value by the disfluent-time value and assigning the result to a fluent to disfluent ratio (FTDR).

39. The device of claim 33, wherein detecting a disfluent speech time-interval(s) in the speech period comprises detecting a time-interval in the speech period in which there is an unnecessary/redundant repetitiveness of a sound, syllable, part of a word, word and/or phrase.

40. The device of claim 33, wherein detecting a disfluent speech time-interval(s) in the speech period comprises detecting a time-interval that includes an intermittent vocal utterance or interjection.

41. The device of claim 33, wherein detecting a disfluent speech time-interval(s) in the speech period comprises detecting a time-interval that includes an abrupt vocal utterance.

42. The device of claim 33, wherein detecting a disfluent speech time-interval(s) in the speech period comprises detecting a time-interval that includes a prolongation having a duration that exceeds a predetermined threshold.

43. The device of claim 33, wherein detecting a disfluent speech time-interval(s) in the speech period comprises detecting a time-interval that includes blocking of speech.

44. The device of claim 33, wherein said processing circuitry is further configured to convert the electrical signal of speech to a frequency domain and to detect a disrupted/stuttered speech time-interval(s) in the speech period by analyzing the electrical signal in the frequency domain.

45. The device of claim 33, wherein said processing circuitry is further configured to calculate a progression score by comparing the derived speech efficiency score with a reference speech efficiency.

46. The device of claim 33, wherein said processing circuitry is configured to perform an offline analysis, such that the steps of detecting the disfluent speech time-interval(s), calculating the disfluent-time value, detecting the fluent speech time-interval(s), calculating the fluent-time value, and deriving a speech efficiency score of the speech period, are performed after the speech period is expired.

47. The device of claim 33, wherein said processing circuitry is configured to perform an online analysis, such that the steps of detecting the disfluent speech time-interval(s), calculating the disfluent-time value, detecting the fluent speech time-interval(s), calculating the fluent-time value, and deriving a speech efficiency score of the speech period, are at least partially performed before the speech period is expired.

48. The device of claim 33, further comprising a user interface unit configured to provide the user with information related to a speech.

49. The device of claim 48, wherein the user is a speaker and/or a practitioner.

50. The device of claim 33, wherein said processing circuitry is configured to derive a speech efficiency score of the speech period by dividing the fluent-time value with the sum of the fluent-time value and disfluent-time.

51. A speech fluency assessment/evaluation method, comprising:
determining a speech period;
obtaining an electrical signal of speech within the speech period;
detecting a disfluent speech time-interval(s) in the speech period, and calculating a disfluent-time value based thereon;
detecting a fluent speech time-interval(s) in the speech period, and calculating a fluent-time value based thereon; and
deriving a speech efficiency score of the speech period based on the fluent-time value and the disfluent-time value.

52. The method of claim 51, further comprising:
detecting an active speech time-interval(s) in the speech period; and
calculating the fluent-time value, calculating the disfluent-time value and deriving the speech efficiency score within the active speech time-interval(s) of the speech period.
